(19) United States
(12) Patent Application Publication
(10) Pub. No.: US 2023/0039887 A1
(43) Pub. Date: Feb. 9, 2023

(54) COMPOSITIONS FOR TOPICAL TREATMENT OF RADIATION DERMATITIS

(71) Applicant: Enveric Biosciences, Inc., Naples, FL (US)

(72) Inventors: Stella K. Vnook, Churchville, PA (US); Daniel J. Kaline, Grand Rapids, MI (US); Ramanathan Lalgudi, Westerville, OH (US)

(21) Appl. No.: 17/869,689

(22) Filed: Jul. 20, 2022

Related U.S. Application Data
(60) Provisional application No. 63/321,260, filed on Mar. 18, 2022.
(60) Provisional application No. 63/223,697, filed on Jul. 20, 2021.

(51) Int. Cl. A61K 31/05; A61K 47/44; A61K 9/00

(52) U.S. Cl. CPC A61K 31/05; A61K 47/44; A61K 9/0014

(57) ABSTRACT

The present disclosure relates to topical compositions and methods for the treatment of radiation dermatitis. The compositions include a cannabinoid and an oil comprising at least 1 wt % of at least one molecule containing an aliphatic chain with at least one cyclopropyl or cyclopropenyl group, such as a cyclopropene fatty acid or a cyclopropane fatty acid. The compositions can also include other components such as stabilizers, thinners, thickeners, fragrances, moisturizers, emollients; and components that provide anti-irritation or anti-inflammatory properties to the compositions.

### CROSS-REFERENCE TO RELATED APPLICATIONS

This application claims priority to U.S. Provisional Patent Application Ser. No. 63/321,260, filed on Mar. 18, 2022, and to U.S. Provisional Patent Application Ser. No. 63/223,697, filed on Jul. 20, 2021, which are incorporated by reference in their entireties.

### BACKGROUND

The present disclosure relates to compositions, treatments, and methods for treating a patient with radiation dermatitis or prophylactically prior to developing radiation dermatitis. This is done by administering, in therapeutically effective amounts, a composition comprising a cannabinoid and an oil containing at least 0.1 wt % of a cyclopropene fatty acid or cyclopropane fatty acid (CPFA), or in more specific embodiments, cannabidiol (CBD) and baobab oil.

Radiation treatment is used to treat malignancies, and is also used during interventional procedures such as coronary angiography, embolization procedures, and indwelling catheter placements. Of particular interest to doctors engaged in radiation therapy is the occurrence and management of radiation dermatitis, which is also known as radiodermatitis, x-ray dermatitis, radiation skin damage, or a radiation burn. Radiation dermatitis is a side effect of external beam ionizing radiation, commonly applied for example during treatments for cancer and other disorders. The radiation can deposit energy at the surface of the skin or several centimeters below the skin surface, depending on where the maximum dose is deposited.

Radiation dermatitis generally manifests within a few days to weeks after the start of radiotherapy. Symptoms can include changes ranging from faint erythema (reddening) during the first 2 weeks of treatment before progressing to desquamation (peeling skin) to skin necrosis (death of skin cells) and ulceration, depending on the severity of the reaction. Other changes in the skin can include the disappearance of follicular structures (pores); an increase in collagen and damage to elastic fibers in the dermis; a fragile surface skin (epidermis); and telangiectasia (prominent blood vessels).

Current treatments for radiation dermatitis include washing the affected skin with a gentle non-soap cleanser and drying with a soft, clean towel before each irradiation session. Emollients, moisturizers, gels, emulsions, and dressings, for example Aquaphor® or Eucerin®, can be applied after treatment and may reduce discomfort. Topical corticosteroids are sometimes prescribed for radiation dermatitis for 2 to 4 weeks.

### BRIEF DESCRIPTION

There remains a medical need for additional safer and more efficient formulations for the treatment and management of radiation dermatitis. Disclosed in various embodiments herein are topical pharmaceutical formulations which comprise a cannabinoid and an oil containing at least 0.1 wt % of a molecule containing an aliphatic chain with a cyclopropyl or cyclopropenyl group, such as a cyclopropene fatty acid or cyclopropane fatty acid (CPFA). In more specific embodiments, the formulations comprise cannabidiol (CBD) and baobab oil. Methods for the treatment of radiation dermatitis using such formulations and compositions are also described herein.

These and other non-limiting aspects of the disclosure are more particularly set forth below.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 is a bar graph showing cell viability for several different formulations containing CBD and baobab oil, along with other formulations for comparative purposes.

### DETAILED DESCRIPTION

The present disclosure may be understood more readily by reference to the following detailed description of desired embodiments and the examples included therein. In the following specification and the claims which follow, reference will be made to a number of terms which shall be defined to have the following meanings.

Although specific terms are used in the following description for the sake of clarity, these terms are intended to refer only to the particular structure of the embodiments selected for illustration in the drawings, and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

The term “comprising” is used herein as requiring the presence of the named components/steps and allowing the presence of other components/steps. The term “comprising” should be construed to include the term “consisting of”, which allows the presence of only the named components/steps. The term “consisting essentially of” should be construed to require the presence of the named ingredients/components/steps, and also permit the presence of other ingredients/components/steps that do not materially affect the basic and novel characteristics of the composition. For purposes of this disclosure, the basic and novel characteristics of the composition are its ability to treat radiation dermatitis.

Numerical values should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values). The endpoints of the ranges and any values disclosed herein are not limited to the precise range or value; they are sufficiently imprecise to include values approximating these ranges and/or values.

The modifier “about” used in connection with a quantity is inclusive of the stated value and has the meaning dictated by the context. When used in the context of a range, the modifier “about” should also be considered as disclosing the range defined by the absolute values of the two endpoints. For example, the range of “from about 2 to about 10” also discloses the range “from 2 to 10.” The term “about” may refer to plus or minus 10% of the indicated number. For example, “about 10%” may indicate a range of 9% to 11%, and “about 1” may mean from 0.9-1.1.

The phrase “therapeutically effective amount” means a sufficient amount of the compound to treat disorders, at a reasonable benefit/risk ratio applicable to any medical treatment. It will be understood, however, that the total daily usage of the compositions of the present disclosure will be decided by the attending physician or other care provider within the scope of sound medical judgment. The specific therapeutically effective dose level for any particular patient will depend upon a variety of factors, including the disorder being treated and the severity of the disorder; medical history of the patient, age, body weight, general health, sex and diet of the patient, the time of administration, route of administration, the duration of the treatment, other drugs being taken by the patient, and the like. A single administration may be sufficient to produce a therapeutic effect, but it is contemplated that multiple administrations will be used over a substantial period of time to assure continued response.

The term “up to X” is used in this disclosure to indicate an amount of an ingredient. This term should be construed to require the ingredient to be present in an amount greater than zero, or in other words to exclude the value zero.

The term “room temperature” means a temperature from 20° C. to 25° C.

The term “oil” refers to an organic substance which is liquid at room temperature that is both hydrophobic and lipophilic. The substance may be a mixture of several different compounds.

The present disclosure relates to compositions and methods for the prophylaxis and treatment of dermatological conditions, especially radiation dermatitis which may arise after exposure to radiation, whether intentional (e.g. during medical treatment) or un-intentional. The compositions are intended to be applied topically. The compositions include (A) a cannabinoid; and (B) an oil comprising at least 0.1 wt % of at least one molecule containing an aliphatic chain with at least one cyclopropyl or cyclopropenyl group. The oil acts as a carrier or solubilizer for the cannabinoid. In further embodiments, the compositions include at least two ingredients: (A) cannabidiol, also known as (CBD); and (B) baobab oil as the carrier for the CBD.

Cannabinoid

The compositions of the present disclosure include (A) a cannabinoid, which is non-psychoactive. A cannabinoid is a compound derived from the cannabis plant, also known as cannabis or hemp. Over 60 different cannabinoids are known to be naturally present in the cannabis plant. Cannabinoids can also be found in other plants such as, without limitation, rhododendron, licorice, liverwort, echinacea, clove, black pepper, broccoli, ginseng carrots, sunflowers, electric daisy, kava, tea plants, cacao, and black truffles. Cannabinoids can also be synthetically produced and are commercially available, for example from PureForm Global (Los Angeles, Calif.). Synthetic cannabinoids which bind to the CB₁ receptor are also known, such as benzoylindoles, cyclohexylphenols, naphthoylindoles, and phenylacetylindoles. Some non-limiting examples of cannabinoids include cannabigerolic acid, cannabigerol, cannabinols, and cannabidiols.

The cannabinoid can be any cannabinoid or derivative thereof. For example, the cannabinoid can be a cannabidiol, cannabigerol, cannabichromene, tetrahydrocannabinol, cannabicyclol, cannabielsoin, cannabinol, cannabinodiol, cannabitriol, dehydrocannabifuran, cannabifuran, cannabichromanon, or cannabiripsol.

Examples of cannabidiols include cannabidiolic acid (CBDA), cannabidiol (CBD), cannabidiol monomethylether (CBDM), cannabidiol-C4 (CBD-C4), cannabidivarinic acid (CBDVA), cannabidivarin (CBDV), and cannabidiorcol (CBD-Ci).

Examples of cannabigerols include cannabigerolic acid (CBGA), cannabigerolic acid monomethylether (CBGAM), cannabigerol (CBG), cannabigerol monomethyleither (CBGM), cannabigerovarinic acid (CBGVA), and cannabigerovarin (CBGV).

Examples of cannabichromenes include cannabichromenic acid (CBC), cannabichromene (CBC), cannabichromevarinic acid (CBCVA), and cannabichromevarin (CBCV).

Examples of tetrahydrocannabinols include D-9-tetrahydrocannabinolic acid A (THCA-A), D-9-tetrahydrocannabinolic acid B (THCA-B), D-9-tetrahydrocannabinol (THC), D-9-tetrahydrocannabinolic acid-C4 (THCA-C4), A-9-tetrahydrocannabinol-C4 (THC-C4), D-9-tetrahydrocannabivarinic acid (THCVA), D-9-tetrahydrocannabivarin (THCV), D-9-tetrahydrocannabiorcolic acid (THCA-Ci), D-9-tetrahydrocannabiorcol (THC-Ci), D-7-cis-tetrahydrocannabivarin, D-8-tetrahydrocannabinolic acid (A⁸-THCA), and D-8-tetrahydrocannabinol (A⁸-THC).

Examples of cannabicyclols include cannabicyclolic acid (CBLA), cannabicyclol (CBL), and cannabicyclovarin (CBLV).

Examples of cannabielsoins include cannabielsoic acid A (CBEA-A), cannabielsoic acid B (CBEA-B), and cannabielsoin (CBE).

Examples of cannabinols and cannabinodiols include cannabinolic acid (CBNA), cannabinol (CBN), cannabinol-C4 (CBN-Cr), cannabivarin (CBV), cannabinol-C2 (CBN-C2), cannabiorcol (CBN-Ci), cannabinodiol (CBND), and cannabinodivarin (CBVD).

Examples of cannabitriols include cannabitriol (CBT), 10-ethoxy-9-hydroxy-A-6a-tetrahydrocannabinol, cannabitriolvarin (CBTV), and ethoxy-cannabitriolvarin (CBTYE).

Cannabifurans include dehydrocannabifuran (DCBF) and cannabifuran (CBF).

Examples of other cannabinoids include cannabichromanon (CBCN), 10-oxo-A-6a-tetrahydrocannabinol (OTHC), cannabiripsol (CBR), and trihydroxy-D-9-tetrahydrocannabinol (triOH-THC).

Derivatives of such cannabinoids may be used as well. For example, the hydroxyl, methyl, and olefinic groups can be modified to enhance binding affinity to cannabinoid receptors CB1 or CB2. For example, the groups may be modified through acetylation, methylation, or neutralization to form salts. Examples of such derivatives are illustrated in structures (CM-1) through (CM-5):

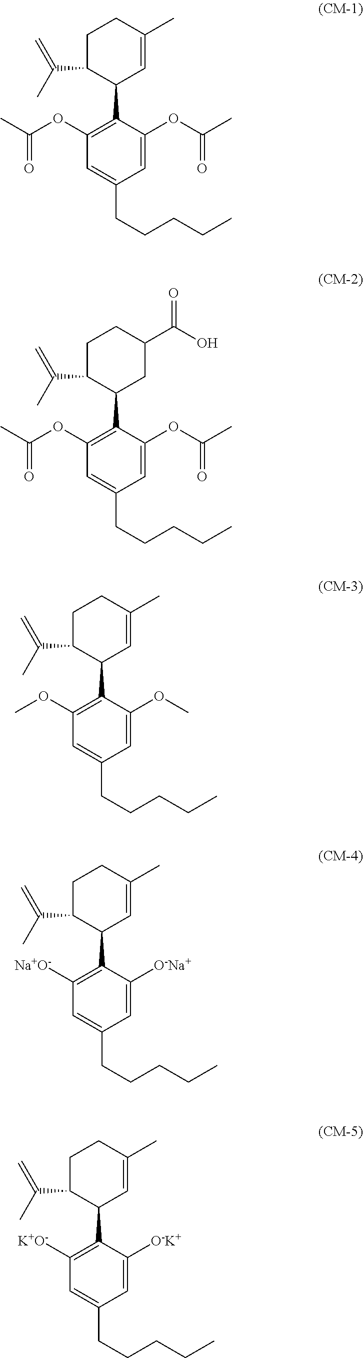

The hydroxyl groups in derivatives (CM-1) and (CM-2) are acetylated, and (CM-2) is also carboxylated. The hydroxyl groups in derivative (CM-3) have been methylated. The sodium and potassium salts of CBD are illustrated in derivatives (CM-4) and (CM-5). Other salts, such as lithium or cesium, are also contemplated.

In particular embodiments, the cannabinoid is a cannabidiol or a cannabidiol derivative. As used herein, the terms “cannabidiol” and “CBD”, as used herein, are intended to refer to the compound 2-[(1R,6R)-6-Isopropenyl-3-methylcyclohex-2-en-1-yl]-5-pentylbenzene-1,3-diol, which is also designated as CAS #13956-29-1. CBD is a major non-psychoactive constituent of cannabis, in contrast to delta-9-tetrahydrocannabinol (THC) which is limited in therapeutic applications due to its psychoactive effects.

CBD can be provided as “full-spectrum CBD,” “broad spectrum CBD,” or “CBD isolate.”

“Full-spectrum CBD” includes cannabinoids, terpenes, and flavonoids, as well as other less plentiful components (sterols, thiols, phenols, lipids or waxes, and fibrous materials). Full-spectrum CBD from cannabis includes CBD and THC as well as more than 100 minor cannabinoids present in small amounts; more than 150 terpenes which affect the plant's fragrance; and about 20 different flavonoids. Full-spectrum CBD can contain up to 0.3% THC.

In comparison, “broad-spectrum CBD” includes the same ingredients as full-spectrum CBD, except THC has been specifically removed. “CBD isolate” is only CBD. These materials can be provided in liquid form or powder form, and can be produced using conventional manufacturing processes.

It is noted that both CBD and THC are derived from cannabigerol (CBG), which itself is a cannabinoid and is one of the higher concentration constituents of full-spectrum CBD and broad-spectrum CBD.

Synthetic CBD is chemically produced in the laboratory, and as such is also free of THC and other plant compounds. Such laboratory manufacturing processes are conventional. Synthetic CBD and CBD isolate may be generally referred to separately or together as synthetic CBD.

Some examples of cannabidiol derivatives may include cannabidiol acetate and cannabidiol-2′,6′-dimethyl ether. Other examples of cannabidiol derivatives may include conjugate molecules formed from an active agent which is linked to a cannabinoid moiety through a specified linker, such as the steroid-cannabinoid conjugates described in PCT publications WO 2020/263888 A1 and WO 2021/076197 A1.

The cannabinoid is present in the compositions/formulations of the present disclosure in a pharmaceutically effective amount. In particular embodiments, the cannabinoid is present in concentrations of from about 3 wt % to about 25 wt %, i.e. about 30 milligram per milliliter (mg/mL) to about 250 mg/mL. In further embodiments, cannabinoid is present in concentrations of from about 0.1 wt % to about 25 wt %, i.e. about 1 milligram per milliliter (mg/mL) to about 200 mg/mL. In more specific embodiments, cannabinoid is present in concentrations of from about 0.1 wt % to about 20 wt %, or from about 0.1 wt % to about 15 wt %, or about 0.1 wt % to about 10 wt %, or about 0.1 wt % to about 5 wt %, or about 1 wt % to about 20 wt %, or about 1 wt % to about 15 wt %, or about 1 wt % to about 10 wt %, or about 1 wt % to about 5 wt %. Such concentrations may be suitable for use in cosmeceuticals or nutraceuticals.

In other embodiments, the cannabinoid may be present in the compositions/formulations of the present disclosure in concentrations ranging from 1 milligram per milliliter (mg/mL) to about 750 mg/m L. In more specific embodiments, the concentration of the cannabinoid may be from about 30 mg/mL to about 750 mg/mL, or from about 60 mg/mL to about 750 mg/mL, or from about 60 mg/mL to about 120 mg/mL, or from about 60 mg/mL to about 500 mg/mL, or from about 500 mg/mL to about 750 mg/mL.

In other embodiments, the CBD is present in concentrations of about 30 mg/mL to about 250 mg/mL, or about 30 mg/mL, or about 60 mg/mL, or about 90 mg/mL, or about 120 mg/mL, or about 150 mg/mL, or about 180 mg/mL, or about 210 mg/mL, or about 250 mg/mL, or in ranges bounded by any two of these values. The cannabinoid in the compositions/formulations of the present disclosure is preferably a cannabinoid derived from natural products having no or low THC content, such as that produced by PureForm Global, Inc., Los Angeles, Calif.

Oil/Baobab Oil

The oil may be present in the amount of about 1 wt % to about 99.9999 wt % of the composition, including from about 40 wt % to about 99.9999 wt %. The oil may alternatively be present in the amount of greater than 50 wt % to about 99.9999 wt %, or from about 50 wt % to about 90 wt %, or greater than 50 wt %, or greater than 60 wt %, or greater than 70 wt %, or greater than 80 wt %, or greater than 90 wt %, or about 90 wt % to about 99.9999 wt %, or from greater than 50 wt % to about 95 wt %, or up to about 99 wt %, or up to about 98 wt % of the composition. In more specific contemplated embodiments, the oil is from about 5 wt % to about 10 wt % of the composition or formulation. In other embodiments, the oil is present in an amount of up to 27.75 wt % of the composition, including about 20 wt % to about 27 wt %. or about 5 wt %, or about 6 wt %, or about 8 wt %, or about 10 wt %, or about 12 wt %, or about 14 wt %, or about 15 wt %, or about 16 wt %, or about 18 wt %, or about 20 wt %, or about 22 wt %, or about 24 wt %, or about 25 wt %, or ranges bounded by any two of these values.

In some embodiments, the oil is baobab oil. Baobab oil is the oil extracted from baobab seeds (CAS #91745-12-9; 225233-93-2). Baobab oil includes high amounts of omega-3 fatty acids, palmitic acid, oleic acid, linolenic acid, and linoleic acid. Baobab oil also includes vitamin C, vitamin B, and minerals such as calcium, iron, potassium, magnesium, phosphorus, and manganese. It is reported to have anti-oxidant, anti-inflammatory, and wound healing properties. However, baobab oil is not currently identified by the US Food and Drug Administration (FDA) as being Generally Recognized As Safe (GRAS). Baobab oil is reported to have a density of 0.85 to 0.95 g/cc.

Baobab oil is believed to contain the following fatty acids in the approximate listed ranges (based on total weight of the oil) in Table 1:

TABLE 1

| | | Amount |
|---|---|---|
| Fatty acid | CAS No. | (wt %) |
| Palmitic Acid (16:0) | 57-10-3 | about 18 to about 48 |
| Palmitoleic (16:1) | 373-49-9 | about 0.1 to about 2 |
| Stearic acid (18:0) | 57-11-4 | about 1 to about 6 |
| Arachidic Acid (20:0) | 506-30-9 | about 0.2 to about 2 |
| Oleic acid (18:1) | 112-80-1 | about 13 to about 44 |
| Linoleic acid (18:2) | 60-33-3 | about 11 to about 37 |
| Linolenic acid (18:3) | 463-40-1 | 0 to about 3 |
| Vaccenic acid | 506-17-2 | about 0.5 to about 3 |
| Sterculic acid | 738-87-4 | about 0.4 to about 6 |
| Dihydrosterculic acid | 5711-28-4 | about 1.5 to about 12 |
| Heptadecenoic acid | 29743-97-3 | about 0.1 to about 2 |
| Malvalic acid | 503-05-9 | about 1 to about 10 |

Baobab oil can be used in the compositions/formulations of the present disclosure as a carrier for the cannabinoid/CBD.

More generally, the oil used in the formulations of the present disclosure comprise at least 0.1 wt % of at least one molecule containing an aliphatic chain with at least one cyclopropyl or cyclopropenyl group. In more particular embodiments, the oil comprises from at least 0.1 wt % to about 12 wt % of the at least one molecule, or at least 0.1 wt % to about 10 wt % of the at least one molecule, including from about 0.5 wt % to about 5 wt %, or from about 1 wt % to about 4 wt %, or a range made from any combination of two of these listed endpoints. Each molecule may be present in an amount within these ranges, or the total amount of such molecules in the oil may be within these ranges.

The term “aliphatic” refers to a hydrocarbon chain which is linear or branched, and which contains at least eight carbon atoms. The carbon atoms of the cyclopropyl or cyclopropenyl group(s) are counted towards the number of carbon atoms in the hydrocarbon chain. The hydrogen atoms on the hydrocarbon chain may be substituted with heteroatom-containing groups such as hydroxyl (—OH) or others, so long as the overall chain remains nonpolar and the molecule is insoluble in water.

Such molecules containing an aliphatic chain with at least one cyclopropyl or cyclopropenyl group may be natural or synthetic. Non-limiting examples of such molecules include sterculic acid, malvalic acid, and dihydrosterculic acid. Such molecules could be artificially synthesized, for example, by reacting an epoxy-containing molecule with a carboxylic acid containing a cyclopropyl group. The carboxylic acid group would react with the epoxy group to obtain a product that contains a hydroxyl group and the cyclopropyl group. In some embodiments, the molecule contains one or two cyclopropyl or cyclopropenyl groups. In particular embodiments, each aliphatic chain contains up to 24 carbon atoms.

In particular embodiments, the molecule containing an aliphatic chain with at least one cyclopropyl or cyclopropenyl group is a cyclopropene fatty acid or a cyclopropane fatty acid (collectively a CPFA). Such fatty acids include a carboxylic acid at the end of an aliphatic chain. The fatty acid can be present in the form of a free fatty acid, or as an ester such as a triglyceride, phosopholipid, or a cholesteryl ester. The cyclopropene fatty acid(s) are generally mono-unsaturated fatty acids (MUFA), and not poly-unsaturated fatty acids. The cyclopropane fatty acid(s) are generally saturated fatty acids. It is contemplated that the presence of CPFAs may improve the properties of the oil as a carrier for the cannabinoid. Thus, the addition of one or more CPFAs to an oil may result in a “synthetic” oil that mimics the performance of baobab oil.

In more particular embodiments, the oil comprises from at least 0.1 wt % to about 12 wt % of the at least one CFPA, or at least 0.1 wt % to about 10 wt % of the at least one CFPA, including from about 0.5 wt % to about 5 wt %, or from about 1 wt % to about 4 wt %, or a range made from any combination of two of these listed endpoints. Each CPFA may be present in an amount within these ranges, or the total amount of CPFAs in the oil may be within these ranges.

In particular embodiments, the CPFA is sterculic acid and/or malvalic acid and/or dihydrosterculic acid. As noted above, these three CPFAs are present in baobab oil. Sterculic acid is 8-(2-octylcycloprop-1-en-1-yl)octanoic acid, and contains a 9,10-cyclopropenyl group. Malvalic acid is 7-(2-octylcycloprop-1-en-1-yl)heptanoic acid, and contains an 8,9-cyclopropenyl group. Dihydrosterculic acid is similar to sterculic acid, but contains a 9,10-cyclopropyl group instead. In some particular embodiments, sterculic acid is present in an amount of about 0.4 wt % to about 6 wt % of the oil, or from about 0.4 to about 2 wt % of the oil. In other particular embodiments, malvalic acid is present in an amount of about 1 wt % to about 10 wt % of the oil, or from about 1 wt % to about 5 wt % of the oil. In still further particular embodiments, dihydrosterculic acid is present in an amount of from about 1.5 wt % to about 12 wt % of the oil, including from about 1.5 wt % to about 5 wt % of the oil. Any combination of two of these acids, or all three of these acids, may also be present in the oil, in an amount as listed.

The oil may further comprise vaccenic acid and/or heptadecenoic acid. Vaccenic acid is (11E)-octadec-11-enoic acid. In some embodiments, vaccenic acid is present in an amount of about 0.5 wt % to about 3 wt % of the oil. In other embodiments, heptadecenoic acid is present in an amount of about 0.1 wt % to about 2 wt % of the oil. Both of these acids may also be present in the oil, in an amount as listed.

It is contemplated that any combination of the CPFAs, along with vaccenic acid, and/or heptadecenoic acid, could be added to another “precursor” oil, such as soybean oil or olive oil, or cottonseed oil, almond oil, apricot kernel oil, castor oil, coconut oil, lanolin oil, lavender oil, or spearmint oil. These oils are believed to contain the following fatty acids in the approximate listed ranges (based on total weight of the oil) in Tables 2A-2D:

TABLE 2A

| | Amount in soybean oil | Amount in olive oil |
|---|---|---|
| Fatty acid | (wt %) | (wt %) |
| Palmitic Acid (16:0) | about 7 to about 13.3 | about 7.5 to about 20 |
| Palmitoleic (16:1) | — | about 0.3 to about 3.5 |
| Stearic acid (18:0) | about 2 to about 6 | about 0.5 to about 5 |
| Arachidic Acid (20:0) | 0 to about 3 | 0 to about 0.8 |
| Oleic acid (18:1) | about 17 to about 30 | about 55 to about 83 |
| Linoleic acid (18:2) | about 50 to about 60 | about 3.5 to about 21 |
| Linolenic acid (18:3) | about 2 to about 13 | 0 to about 1.5 |

TABLE 2B

| | Amount in cottonseed | Amount in almond oil |
|---|---|---|
| Fatty acid | oil (wt %) | (wt %) |
| Palmitic Acid (16:0) | about 11.7 to about 26.4 | about 4.3 to about 15.8 |
| Palmitoleic (16:1) | 0 to about 1.2 | about 0.1 to about 6 |
| Stearic acid (18:0) | about 1.5 to about 9.9 | about 0.2 to about 10.4 |
| Arachidic Acid | about 0.1 to about 1.3 | 0 to about 0.5 |
| (20:0) | | |
| Oleic acid (18:1) | about 14.7 to about 29.4 | about 43.3 to about 81.6 |
| Linoleic acid (18:2) | about 13.1 to about 58.3 | about 6.2 to about 37.1 |
| Linolenic acid | 0 to about 0.9 | 0 to about 1.4 |
| (18:3) | | |
| Vaccenic acid | 0 to about 0.87 | about 0.7 to about 2.2 |
| Sterculic acid | about 0.08 to about 0.33 | — |
| Malvalic acid | about 0.12 to about 0.42 | — |
| eicosenoic acid | 0 to about 27.6 | 0 to about 0.3 |
| (20:1) | | |

TABLE 2C

| | Amount in apricot kernel | Amount in castor oil |
|---|---|---|
| Fatty acid | oil (wt %) | (wt %) |
| Palmitic Acid (16:0) | about 2.9 to about 7.6 | about 1 to about 2 |
| Palmitoleic (16:1) | about 0.3 to about 0.8 | — |
| Stearic acid (18:0) | about 0.9 to about 2.0 | about 1 to about 2 |
| Arachidic Acid (20:0) | about 0.1 to about 0.3 | — |
| Oleic acid (18:1) | about 41.1 to about 74.8 | about 3 to about 4 |
| Linoleic acid (18:2) | about 17.4 to about 45.6 | about 4 to about 5 |
| Linolenic acid (18:3) | 0 to about 1.4 | about 0.5 to about 0.7 |
| Vaccenic acid | — | — |
| Sterculic acid | — | — |
| Malvalic acid | — | — |
| Heptadecenoic acid | — | 0 to about 0.1 |
| eicosenoic acid (20:1) | 0 to about 0.2 | — |
| ricinoleic acid | — | about 85 to about 90 |

TABLE 2D

| | Amount in coconut oil | Amount in lavender |
|---|---|---|
| Fatty acid | (wt %) | oil (wt %) |
| Palmitic Acid (16:0) | — | about 5.4 to about 5.6 |
| Palmitoleic (16:1) | 0.1 to about 0.2 | 0 to about 0.1 |
| Stearic acid (18:0) | 1 to about 4 | 2.2 to about 2.4 |
| Arachidic Acid (20:0) | 0.1 to about 0.4 | 0.1 to about 0.2 |
| Oleic acid (18:1) | 5 to about 8 | 6.8 to about 7.6 |
| Linoleic acid (18:2) | — | 17.8 to about 18.3 |
| Linolenic acid (18:3) | 1.1 to about 2 | 65.2 to about 67.3 |
| Vaccenic acid | — | — |
| Sterculic acid | — | — |
| Malvalic acid | — | — |
| caproic acid | 0.5 to about 0.8 | — |
| caprilic acid | 5 to about 9 | — |
| capric acid | 4 to about 10 | — |
| lauric acid | 44 to about 52 | — |
| myristic acid | 8 to about 21 | — |

Mineral oil could also be used as a “precursor” oil. The CPFA(s), along with any combination of dihydrosterculic acid, vaccenic acid, and/or heptadecenoic acid, would be added to the “precursor” oil to obtain an amount corresponding to the ranges described above in Table 1 for each fatty acid, to produce a synthetic oil that mimics baobab oil.

In particular embodiments, the synthetic oil may contain up to 15 wt % of palmitic acid; and/or from 50 wt % to about 80 wt % oleic acid; and/or up to 8 wt % of linoleic acid, including any combination of one of these ranges, or two of these ranges, or three of these ranges. The synthetic oil may also contain from about 2 wt % to about 10 wt % of stearic acid.

In some other embodiments, the oil can be present in an amount from about 75 wt % to about 97 wt % of the composition/formulation.

Additional Ingredients

The topical compositions/formulations of the present disclosure may include other ingredients besides the cannabinoid and the oil (which can be baobab oil). Such ingredients may include comprise mineral oil, petrolatum, medium-chain triglycerides (MCT oil), olive oil, sunflower oil and other components that provide structure or serve as stabilizers, thinners, thickeners, fragrances, moisturizers, emollients and/or ingredients that provide additional anti-irritation or anti-inflammatory properties to the formulation. Some such ingredients are described in this section.

When present, the mineral oil is desirably an NF grade light mineral oil, which will not clog the pores of the skin. United States Pharmacopeia (USP) standards should be met.

Petrolatum, aka “petroleum jelly,” is useful in topical ointments as a carrier or base, as opposed to water-based cremes or gels. Petrolatum is known to temporarily protect minor cuts, scrapes and burns, and for being effective in keeping skin moist during post-surgery healing. Petrolatum comes in many grades. To the extent that the formulations of the present disclosure uses petrolatum, pharmacy grade petrolatum will be used, such as white petrolatum USP meeting requirements for USP and FDA requirements of 21 CFR 172.880, which are commercially available.

Fragrances, moisturizers, and emollients may also be variously and preferably included in the formulations disclosed herein, with fragrances typically at concentrations less than about 1 wt %.

Examples of additional emollients and oils that can be included in the topical formulation are olive oil, medium-chain triglycerides (MCT oil), sunflower oil, long-chain triglycerides (LCT oil), long-chain fatty acids (e.g., myristic acid, palmitic acid, stearic acid, arachidic acid, linoleic acid), glycerine/glycerol, glycerol monolinoleate, coconut oil, corn oil, canola oil, avocado oil, vegetable oil, flaxseed oil, palm oil, palm kernel oil, peanut oil, rice bran oil, safflower oil, jojoba oil, argan oil, grapeseed oil, castor oil, wheat germ oil, peppermint oil, and sesame oil. It is noted that medium-chain triglycerides are those in which the carbohydrate chain has 8 to 12 carbon atoms. Each of these additional oils can be present in the amount of from about 1 wt % to about 99.9999 wt %. The total amount of these additional oils may be from about 1 wt % to about 99.9999 wt %.

Synthesis/Preparation

The compositions/formulations of the present disclosure can be made using methods known in the art. For example, the composition can be made by mixing the cannabinoid with the oil-phase materials and dissolving the cannabinoid, usually done at room temperature. Any aqueous-phase materials are then combined. The oil-phase mixture and the aqueous-phase mixture are then combined and homogenized to form an emulsion.

Applications

The formulations of the present disclosure are intended to be applied topically to the skin of the user/patient. This results in the active ingredient being delivered to local tissue. The formulations may also operate transdermally, delivering the active ingredient across the skin and into systemic circulation.

The topical formulation may be in the form of a cream, gel, hydrogel, liquid, lotion, or ointment. The topical formulation may be applied manually, sprayed, or by syringe, applicator, or other dispensing means. The topical formulation could alternatively be provided in the form of a matrix-type delivery system, where the formulation is absorbed or suspended in a matrix that is then adhered to a backing membrane (commonly referred to as a bandage or a patch).

The topical formulations of the present disclosure desirably have the advantages of efficacy, safety, tolerability, speed of healing, and lowered sensation of pain for the user/patient.

The formulations may be used to treat, prevent, or ameliorate radiation dermatitis. This is done by applying the topical formulation to the skin of the user/subject/patient. In some embodiments, the topical formulation can be applied for a period of 0 days to 7 days prior to radiation exposure. In another example, the topical formulation is applied after the radiation exposure. The duration of the treatment may be between 24 hours and 14 days. In another example, the duration of the treatment may be between 24 hours and 30 days. In another example, the duration of the treatment is between 1 month to 12 months. With respect to time, the application may be on a regular or irregular schedule, as desired. Generally, the topical formulation is used as needed to ameliorate pain or assist in healing.

In some further embodiments, two different formulations are used together to treat or ameliorate radiation dermatitis. A first formulation comprising a cannabinoid is administered to the patient for a first time period. Subsequently, a second formulation comprising the cannabinoid is administered to the patient for a second time period. The second formulation contains a higher dosage of the cannabinoid than the first formulation. Again, typically, the formulations are made for topical application/administration. The two formulations may be designed to have different permeation rates through the skin, too.

In some specific embodiments, the first formulation contains about 3 wt % of the cannabinoid, and the second formulation contains about 12 wt % of the cannabinoid. In some other specific embodiments, the first formulation contains about 3 wt % to about 12 wt % of the cannabinoid, and the second formulation contains about 12 wt % to about 25 wt % of the cannabinoid (but higher than the first formulation). In other embodiments, the second formulation contains at least 6 wt % (by absolute amt) more of the cannabinoid than the first formulation. For example, if the first formulation contains 3 wt %, the second formulation contains 9 wt % or more. In yet other embodiments, the second formulation contains at least 100% more (relative amt) of the cannabinoid than the first formulation, and up to 400% more. For example, if the first formulation contains 3 wt %, the second formulation contains 6 wt % or more.

The duration of the first time period and the second time period can vary, and do not need to be the same. In some embodiments, the first time period is from about 7 days to about 14 days. In some embodiments, the second time period is from about 7 days to about 14 days. In still other embodiments, the second time period is at least 7 days longer than the first time period.

The present disclosure in a further aspect includes providing a kit comprising the topical composition of the present disclosure; and in a further example providing a kit comprising the topical composition of the present disclosure and any or all of an applicator, such as a pad, utensil, spatula, sprayer or droplet dispenser; and/or a bandage, such as a dermal patch, wrap or other form of bandage and instructions for use thereof.

The present disclosure will further be illustrated in the following non-limiting examples, it being understood that these examples are intended to be illustrative only and that the disclosure is not intended to be limited to the materials, conditions, process parameters and the like recited herein.

### EXAMPLES

Test materials were applied topically for 24 hours and an MTT assay was used to check the viability of cells. The following treatment groups were used:
Baobab oil (no CBD);
CBD in baobab oil in concentrations of 1, 10, 30, 60, 120, 250, 500, and 750 mg/mL;
0.9% saline (no CBD);
No treatment; and
Triton-X® as positive control.

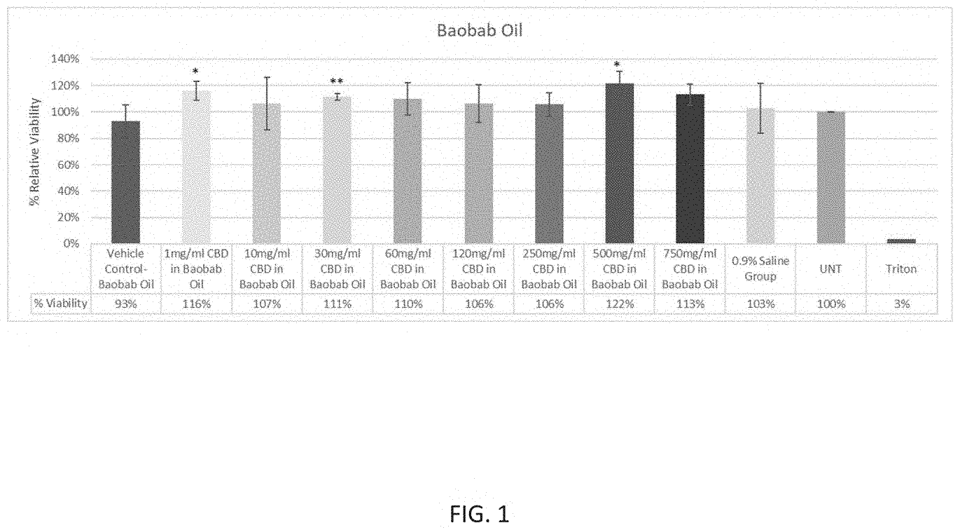

The relative results are provided in the following Table A, with the untreated group defining 100% viability, and shown in FIG. 1. * indicates p≤0.05 and ** indicates p≤0.01 statistical significance after performing unpaired t-test.

TABLE A

| | | Relative Viability |
|---|---|---|
| Label | Treatment | (%) |
| A | Vehicle Control - baobab oil only | 93 |
| B | 1 mg/mL CBD in baobab oil | 116* |
| C | 10 mg/mL CBD in baobab oil | 107 |
| D | 30 mg/mL CBD in baobab oil | 111** |
| E | 60 mg/mL CBD in baobab oil | 110 |
| F | 120 mg/mL CBD in baobab oil | 106 |
| G | 250 mg/mL CBD in baobab oil | 106 |
| H | 500 mg/mL CBD in baobab oil | 122* |
| I | 750 mg/mL CBD in baobab oil | 113 |
| J | 0.9% saline | 103 |
| K | Untreated | 100 |
| L | Triton-X | 3 |

There was a significant increase in cell viability at 1 mg/mL, 30 mg/mL, and 500 mg/mL CBD, meaning the cells were more metabolically active and healthy. In addition, based on existing data, it was expected that the cell viability at 120 mg/mL and greater would be at most 90%, so the results for these values were unexpected.

Next, changes in gene expression were measured in tissues treated topically with the formulations (without radiation exposure) using qPCR. This provides evidence that the CBD is being absorbed by the tissues. Five genes were examined: APOB, CSF2, IL24, MT1F, and MT1G. In particular, MT1F and MT1G code for metallothioneins which are involved in the protection of cells from oxidative stress, and have been shown to increase after treatment with CBD.

Statistical significance was determined using unpaired t-test, P<0.05, N=3, and are indicated in bold. The results are provided in Table B in both linear fold change values (FC) and in percent change. It is noted that changes in gene expression in general were only seen in the treatments where CBD was used. The control (i.e. oil alone) did not alter expression in any of the five genes.

TABLE B

| | 1 mg/mL CBD | | 10 mg/mL CBD | | 30 mg/mL CBD | | 60 mg/mL CBD | |
|---|---|---|---|---|---|---|---|---|
| Gene | FC | % Δ | FC | % Δ | FC | % Δ | FC | % Δ |
| APOB | 1.23 | 23% | 1.00 | 0% | −1.19 | −16% | 1.51 | 51% |
| CSF2 | 2.17 | 117% | 2.18 | 118% | −1.23 | −19% | 2.86 | 186% |
| IL24 | 1.55 | 55% | 1.48 | 48% | −3.34 | −70% | −1.13 | −11% |
| MT1F | 2.47 | 147% | 1.40 | 40% | −2.82 | −65% | 2.34 | 134% |
| MT1G | −1.71 | −41% | −1.04 | −4% | 1.92 | 92% | 26.26 | 2526% |
| | 120 mg/mL CBD | | 250 mg/mL CBD | | 500 mg/mL CBD | | 750 mg/mL CBD | |
| Gene | FC | % Δ | FC | % Δ | FC | % Δ | FC | % Δ |
| APOB | −1.10 | −9% | −2.48 | −60% | −22.22 | −96% | −25.64 | −96% |
| CSF2 | 5.48 | 448% | 8.63 | 763% | −1.43 | −30% | 1.93 | 93% |
| IL24 | 1.63 | 63% | 1.59 | 59% | −1.06 | −6% | 1.54 | 54% |
| MT1F | 7.02 | 602% | 22.63 | 2163% | 40.63 | 3963% | 30.71 | 2971% |
| MT1G | 52.86 | 5186% | 135.39 | 13439% | 335.39 | 33439% | 233.23 | 23223% |

MT1F and MT1G showed a dose-dependent increase, with significant fold changes.

The present disclosure has been described with reference to exemplary embodiments. Modifications and alterations will occur to others upon reading and understanding the preceding detailed description. It is intended that the present disclosure be construed as including all such modifications and alterations insofar as they come within the scope of the appended claims or the equivalents thereof.

1. A topical formulation for the treatment of radiation dermatitis, comprising:
a cannabinoid; and
an oil comprising at least 0.1 wt % of at least one molecule containing an aliphatic chain with at least one cyclopropyl or cyclopropenyl group.

2. The topical formulation of claim 1, wherein the oil comprises from at least 0.1 wt % to about 12 wt % of the at least one molecule containing an aliphatic chain with at least one cyclopropyl or cyclopropenyl group.

3. The topical formulation of claim 1, wherein the at least one molecule containing an aliphatic chain with at least one cyclopropyl or cyclopropenyl group is a cyclopropene fatty acid or cyclopropane fatty acid (CPFA).

4. The topical formulation of claim 3, wherein the cyclopropene fatty acid or cyclopropane fatty acid is sterculic acid or malvalic acid.

5. The topical formulation of claim 4, wherein the oil comprises from about 0.4 wt % to about 6 wt % of sterculic acid; or
wherein the oil comprises from about 1 wt % to about 10 wt % of malvalic acid.

6. The topical formulation of claim 3, wherein the cyclopropene fatty acid or cyclopropane fatty acid is dihydrosterculic acid, which is present in an amount or about 1 wt % to about 5 wt % of the oil.

7. The topical formulation of claim 1, wherein the oil further comprises vaccenic acid or heptadecinoic acid.

8. The topical formulation of claim 7, wherein the oil contains from about 0.5 wt % to about 3 wt % of vaccenic acid; or
wherein the oil contains from about 0.1 wt % to about 2 wt % of heptadecinoic acid.

9. The topical formulation of claim 1, wherein the oil further comprises palmitic acid, stearic acid, arachidic acid, oleic acid, or linoleic acid.

10. The topical formulation of claim 9, wherein the oil contains up to 15 wt % of palmitic acid; or
wherein the oil contains from about 1 wt % to about 6 wt % of stearic acid; or
wherein the oil contains from about 0.2 wt % to about 2 wt % of arachidic acid; or
wherein the oil contains from 50 wt % to about 80 wt % of oleic acid; or
wherein the oil contains up to 8 wt % of linoleic acid.

11. The topical formulation of claim 1, wherein the oil is soybean oil, olive oil, cottonseed oil, almond oil, apricot kernel oil, castor oil, coconut oil, lanolin oil, lavender oil, spearmint oil, or mineral oil, to which the at least one molecule containing an aliphatic chain with at least one cyclopropyl or cyclopropenyl group is added.

12. The topical formulation of claim 1, wherein the cannabinoid is a cannabidiol, cannabigerol, cannabichromene, tetrahydrocannabinol, cannabicyclol, cannabielsoin, cannabinol, cannabinodiol, cannabitriol, dehydrocannabifuran, cannabifuran, cannabichromanon, or cannabiripsol, or a derivative thereof, wherein the derivative is an acetylated derivative, a methylated derivative, or a salt of the cannabinoid.

13. The topical formulation of claim 1, containing from about 1 mg/mL to about 750 mg/mL or from about 60 mg/mL to about 120 mg/mL of the cannabinoid; or
containing from about 3 wt % to about 25 wt % of the cannabinoid; or
containing from about 1 wt % to about 99.9999 wt % of the oil.

14. The topical formulation of claim 1, wherein the oil is baobab oil.

15. A method of treating, preventing, or ameliorating radiation dermatitis in a user, comprising:
applying a topical formulation to the skin of the user;
wherein the topical formulation comprises a cannabinoid and an oil comprising at least 0.1 wt % of at least one molecule containing an aliphatic chain with at least one cyclopropyl or cyclopropenyl group.

16. A method for treating a patient exhibiting radiation dermatitis, comprising:
administering to an area exhibiting radiation dermatitis a first formulation for a first time period; and
subsequently administering to the area exhibiting radiation dermatitis a second formulation for a second time period;
wherein the first formulation and the second formulation each comprise a cannabinoid and an oil comprising at least 1 wt % of at least one molecule containing an aliphatic chain with at least one cyclopropyl or cyclopropenyl group; and
wherein the second formulation contains a higher dosage of the cannabinoid than the first formulation.

17. The method of claim 16, wherein the first formulation contains about 3 wt % of the cannabinoid.

18. The method of claim 16, wherein the second formulation contains about 12 wt % to about 25 wt % of the cannabinoid; or
wherein the second formulation contains at least 6 wt % more of the cannabinoid than the first formulation; or
wherein the second formulation contains at least 100% more of the cannabinoid than the first formulation.

19. The method of claim 16, wherein the first time period is from about 7 days to about 14 days; or
wherein the second time period is from about 7 days to about 14 days.

20. The method of claim 16, wherein the first formulation and the second formulation are administered by topical application.
